(19) United States
(12) Patent Application Publication
(10) Pub. No.: US 2007/0233273 A1
(43) Pub. Date: Oct. 4, 2007

(54) Differential Tissue Expander Implant

(76) Inventor: Anthony Francis Connell, Subiaco (AU)

Correspondence Address: PERKINS COIE LLP, P.O. BOX 2168, MENLO PARK, CA, 94026, US

(21) Appl. No.: 10/558,805

(22) PCT Filed: May 26, 2004
(86) PCT No.: PCT/AU04/00693

§ 371 Date: Mar. 6, 2007

(30) Foreign Application Priority Data
May 26, 2003 (AU) 2003902604

(51) Int. Cl. A61F 2/02

(52) U.S. Cl. 623/23.72; 623/23.74

(57) ABSTRACT

A tissue expander implant (10) arranged to be anchored in position within a cavity formed in the body for the controlled expansion of overlying tissue. The tissue expander implant (10) comprises a first expandable envelope (18) defining a first closed chamber (18a), first inflation means for enabling the controlled inflation of the first expandable envelope with a biocompatible fluid, a second expandable envelope (20) located in side-by-side relationship with the first expandable envelope and defining a second closed chamber (20A) and second inflation means for enabling the controlled inflation of the second expandable envelope with a biocompatible fluid. The first and second expandable envelopes have a textured external surface for promoting adhesion to overlying tissue. The first and second expandable envelopes are differentially expandable independently of one another to enable the tissue overlying the first and second expandable envelopes to be expanded to differing degrees, thereby to maintain, or move, a surface feature of the overlying tissue in, or to, a desired position.

### FIELD OF THE INVENTION

This invention relates to a differential tissue expander implant, and in particular to a differential tissue expander implant for use in breast reconstruction surgery.

### BACKGROUND OF THE INVENTION

The incidence of breast cancer in women in the Western world continues to increase at a startling rate. Over the past twenty years the incidence of breast cancer in Australia and the United States has increased from one in 14 women in their lifetime to a current level of one in 11 women. The current incidence of breast carcinoma in Australia is 90 cases per 100,000 which results in 4,200 new cases per year. The incidence is similar in the United States with approximately 75,000 mastectomies being performed per year.

Advances in epidemiology and genetics have now identified high risk genetic forms of breast cancer related to the BRAC1 and BRAC2 genes which result in a 90% lifetime risk of developing breast carcinoma in these women. Fortunately the genetic form of breast cancer accounts for only 5% of all breast cancer patients and conversely 95% of all breast cancer is sporadic in nature.

The increased incidence of breast cancer has fortuitously been matched with improved treatment modalities resulting in increased survival of women with this form of cancer. Over the past twenty years the five year survival rate from breast carcinoma has significantly improved. Breast cancer is now detected earlier and pre-cancerous forms of breast carcinoma have been identified such as ductal carcinoma in-situ and lobular carcinoma in-situ. Early treatment of these pre-invasive forms of breast cancer can be completely curative.

The increased survival rate in breast cancer is the result of earlier detection, improved surgical techniques and advances in adjuvant treatment with radiotherapy and chemotherapy. The increased earlier detection of breast cancer results in an increased percentage of women who can be treated with breast conservation surgery called lumpectomy. Approximately 60% of women can be treated with this form of surgery and therefore do not require total mastectomy.

In women identified as being at high risk of developing breast carcinoma or if genetic testing identifies them as carriers of the BRAC1 or BRAC2 gene, prophylactic mastectomy is often recommended rather than undergoing lifetime screening with mammograms and frequent breast biopsies. Prophylactic mastectomies are performed via a lateral incision extending from the areola to the lateral aspect of the breast with complete excision of the underlying breast gland with preservation of the overlying skin envelope including the nipple-areola complex (NAC), if the sub-areola biopsy is negative for ductal atypia in this area.

In the field of plastic surgery the sub-specialty of breast reconstruction has evolved over the past twenty years with newer techniques being developed to reconstruct the breast either immediately or in a delayed fashion post-mastectomy. There are principally three types of breast reconstruction involving either autologous tissue, a tissue expander/implant or a combination of both.

One type of totally autologous reconstruction is the pedicled or free TRAM flap raised from the lower abdominal region. Another form of autologous reconstruction involves pedicling the latissimus dorsi muscle from the lateral back and tunneling it anteriorly to the mastectomy area. Often there is insufficient volume in this back muscle to match the volume of the original breast and to increase the volume a tissue expander is placed submuscularly primarily and replaced secondarily with a permanent implant.

The second form of breast reconstruction involves placing a sub-muscular tissue expander under the pectoralis major muscle at the time of the mastectomy and sequentially inflating the expander. The expander is inflated in the surgeon's office while the patient is an outpatient with saline injected into the expander port once or twice per week until the appropriate volume to attain symmetry with the opposite breast is attained. Once the appropriate volume has been attained the patient returns to the operating theatre and the tissue expander is removed and replaced with a permanent prosthesis filled with either saline or solid silicone cohesive gel.

The use of tissue expanders to expand tissue to be used in reconstructive surgery is a technique which has been used widely over the past thirty years. The use of tissue expansion in breast reconstruction was first performed by Radovan in the 1970's and since then this form of breast reconstruction has been used extensively world wide for both unilateral and bilateral reconstruction cases. The largest review series on breast reconstruction shows that between 20 to 30% of all reconstructions post-mastectomy use the tissue expander implant form of reconstruction. The remainder use purely autologous reconstruction or a combination of the autologous and expander implant techniques. Advances in tissue expander technology over the past decade have resulted in new expanders being anatomical or teardrop-shaped to match the natural breast appearance, such as the Contour Profile® Tissue Expander manufactured by Mentor, of 201 Mentor Drive, Santa Barbara, Calif. 931111, United States of America and the 133 LV and 133 FV Biodimensional® expanders manufactured by McGhan Limited, of Kilbride Industrial Estate Arklow, County Wicklow, Ireland.

Over the past decade general surgeons have developed the technique of skin sparing mastectomy including preservation of the NAC if the breast carcinoma is not present within 40 mm of the NAC.

The technique of nipple sparing mastectomy is suitable in up to 80% of women with breast carcinoma as less than 20% of women have a tumour within 40 mm of the NAC. Preserving the skin envelope and NAC dramatically improves the aesthetic results of all forms of reconstruction as the reconstruction only needs to provide sufficient volume to fill out the pre-existing breast envelope.

The vast majority of subcutaneous mastectomies are carried out via a lateral incision extending from the lateral edge of the areola laterally to the anterior axillary line. The breast gland is excised following elevation of the skin envelope off the underlying breast gland. At the end of the procedure the breast tissue lying beneath the NAC is specifically biopsied and examined intra-operatively and post-operatively to exclude the possibility of atypical or malignant cells remaining in the retained NAC which may result in a local recurrence of the breast carcinoma.

On occasion the biopsy is positive and the patient requires a small secondary procedure to excise the NAC to ensure complete excision of the atypical or malignant cells. Previously the NAC was excised in all cases of mastectomy because of the concern that atypical cells may be left beneath the NAC and result in a local recurrence. It is now known that preservation of the NAC when the biopsy is negative does not result in an increased risk of developing a local recurrence of breast carcinoma.

A small group of women are suitable for nipple sparing mastectomies who have bilateral pre-malignant breast tissue such as ductal carcinoma in-situ, or have a very strong family history of breast carcinoma, or have one of the two genetic forms of breast cancer. Preserving the NAC bilaterally in this form of mastectomy significantly improves the patient's aesthetic result post-reconstruction.

There are a number of described techniques in reconstructing the NAC if it is required to be excised for oncological reasons. This minor procedure is usually undertaken three to six months post reconstruction and utilizes local tissue for nipple reconstruction and a full thickness graft from the inner thigh for areola reconstruction. After waiting a further month, the new NAC is tattooed to match the colour of the NAC on the opposite side.

The NAC is aesthetically very important for the overall appearance of the breast and is the point of primary focus when the naked breast is first visualized. It is similar to the nose on a person's face and carries a similar aesthetic value to the patient.

Women who elect to have bilateral prophylactic mastectomies tend to be younger and more active than women who develop breast carcinoma and require mastectomy to excise their tumour. These younger patients often elect bilateral submuscular tissue expander and implant reconstruction as they often have insufficient autologous tissue and are not keen to have additional scars on the lower abdomen or the lateral back from the two forms of autologous reconstruction as mentioned.

In addition, bilateral harvest of the latissimus dorsi muscles can place restrictions on sporting activities. These factors influence women in selecting bilateral sub-muscular tissue expander and implant reconstruction such that in the inventor's experience, 60% select this form of reconstruction if they are undergoing bilateral prophylactic mastectomies.

Upon completion of the subcutaneous mastectomy, an anatomical tissue expander is placed beneath the pectoralis major muscle and its size is selected to correspond to the dimensions of the mastectomy defect and the pre-operative measurements of the base diameter of the breast. The sub-pectoral pocket is dissected in all directions. This includes raising a lateral flap of serratus anterior to form the lateral wall of the expander pocket. This lateral flap of serratus anterior is sutured to the lateral edge of the pectoralis major muscle to completely envelope the tissue expander in a sub-muscular pocket.

A tissue expander is placed in the sub-muscular pocket, haemostasis is attained and a suction drain is placed in this area. The tissue expander is partially inflated with saline and is enclosed in the sub-muscular pocket with the lateral opening of the pocket being closed with interrupted sutures. The mastectomy skin flaps are then assessed for their vascularity and if there is no vascular compromise to the edges of the mastectomy wound the wounds are closed directly.

The patient usually remains an in-patient in hospital for a period of three to four days requiring initially intravenous and subsequently oral analgesia and antibiotics. The patient is discharged from hospital when the suction drain is removed and she is next reviewed approximately one week later where the healing of the mastectomy wound is assessed.

Approximately three and a half weeks following the mastectomy the patient returns to the plastic surgeon's office and the injection port is identified and 60 ml of intravenous saline is injected into the port. Tissue expanders currently can have either a remote or an intrinsic port. A remote port usually has a siloxane polymer tube extending from the undersurface or side of the expander which extends out to a siloxane polymer dome with a metal disc in the base, with the injection port usually positioned in the lateral chest wall. The other type of injection port, the intrinsic port, is usually found in the upper pole of the tissue expander and its position is identified by using a special magnet which is initially provided with the expander mechanism.

The normal expansion protocol involves injecting 60 ml of saline twice a week until the appropriate volume has been inflated into the expander sufficient to match the contralateral breast and to match the base diameter of the breast previously excised.

The patient is then returned to the operating theatre three weeks after the expansion process has been completed and then, via re-opening the mastectomy wound, the tissue expander is removed and either a saline filled or solid silicone cohesive gel filled permanent prosthesis is placed within the sub-muscular pocket.

There are a number of complications that can occur using tissue expanders in breast reconstruction such as:
1. haematoma around the tissue expander within the sub-muscular pocket;
2. seroma around the tissue expander within the sub-muscular pocket;
3. infection of the tissue expander,
4. failure of the injection port or perforation of the expander shell via the injection needle resulting in saline leakage and deflation of the tissue expander; and
5. tissue expander migration resulting in an asymmetrical sub-muscular pocket.

### SUMMARY OF THE INVENTION

In a first aspect the present invention provides a tissue expander implant arranged to be anchored in position within a cavity formed in the body for the controlled expansion of overlying tissue. The tissue expander implant comprises a first expandable envelope defining a first closed chamber, first inflation means for enabling the controlled inflation of the first expandable envelope with a biocompatible fluid, a second expandable envelope located in side-by-side relationship with the first expandable envelope and defining a second closed chamber and second inflation means for enabling the controlled inflation of the second expandable envelope with a biocompatible fluid. The first and second expandable envelopes are differentially expandable independently of one another to enable the tissue overlying the first and second expandable envelopes to be expanded to differing degrees, thereby to maintain, or move, at least one surface feature of the overlying tissue in, or to, a desired position.

Preferably, the tissue expander implant further comprises a base portion and a dividing wall extending from the base portion, the dividing wall being positioned between the first and second expandable envelopes such that the first and second expandable envelopes are located on opposite sides of the dividing wall.

Preferably, the dividing wall is inelastic relative to the operatively outermost walls of the first and second expandable envelopes.

Optionally, the dividing wall is common to, and partially defines, both the first and second adjacent closed chambers.

Optionally, the base portion partially defines the first and second adjacent closed chambers.

In certain embodiments, the dividing wall is arranged to be positioned beneath the at least one surface feature, or a desired location of the at least one surface feature, with the first and second expandable envelopes being arranged to be located beneath and substantially on opposite sides of the at least one surface feature and being configured to expand the overlying tissue to a desired degree when in said position.

In certain embodiments, one of the first or second expandable envelopes is configured to have a larger volume than the other.

Preferably, the external tissue-contacting surface of the expander is textured for promoting adhesion to the tissue surrounding the tissue expander implant.

In certain embodiments, the dividing wall is off-centre in relation to the base portion to define first and second expandable envelopes having differing volumes. Preferably, the dividing wall is positioned approximately one third of the breadth of the base portion from one edge of the base portion.

In certain embodiments, the tissue expander implant is a breast expander implant and the overlying tissue is mammary tissue and the surface feature is a nipple-areola complex. The first and second expandable envelopes are configured to undergo controlled expansion independently of one another via the first and second inflation means respectively to control sideways displacement of the nipple-areola complex.

Preferably, the first and second inflation means are resealable injection valves or ports. Optionally, the resealable injection valves or ports are integral with the first and second expandable envelopes. Alternatively, one of the resealable injection valves or ports is remote from either the first or second expandable envelope and the other resealable injection valve or port is integral with the other expandable envelope.

Preferably, the tissue expander implant is integrally moulded from an elastomeric material and the relative elasticity of the first and second expandable envelopes, the dividing wall and the base portion are determined by their relative thicknesses, with the dividing wall and the base portion being thicker than the first and second expandable envelopes. Preferably, the elastomeric material is silicone.

Optionally, the tissue expander implant further comprises a third expandable envelope defining a third closed chamber, third inflation means for enabling the controlled inflation of the third expandable envelope with a biocompatible fluid, wherein the third expandable envelope is located in a side-by-side relationship with at least one of the first and second expandable envelopes and is expandable independently of the first and second expandable envelopes.

Optionally, the tissue expander implant further comprises a fourth expandable envelope defining a fourth closed chamber, fourth inflation means for enabling the controlled inflation of the fourth expandable envelope with a biocompatible fluid, wherein the fourth expandable envelope is located in a side-by-side relationship with at least one of the first, second or third expandable envelopes and is expandable independently of the first, second and third expandable envelopes.

In another aspect the invention provides a method of expanding tissue to control the displacement of a surface feature on the tissue as it is being expanded. The method comprises:

implanting beneath a section of tissue which is arranged to be expanded a tissue expander implant having first and second expandable envelopes located alongside one another;

monitoring the position of the surface feature on the tissue; and

independently expanding the first and second expandable envelopes in response to said monitoring to maintain or migrate the surface feature in or towards a desired position as the overlying tissue is expanded.

Preferably, the method includes repeating the steps of monitoring the position of the surface feature and independently expanding the first and second expandable envelopes with the aim of attaining the desired position of the surface feature at a desired degree of expansion of the tissue.

Preferably, the method includes the initial step of establishing a desired final position of the surface feature and a desired degree of final expansion.

In a tissue expander further comprising a base portion and a dividing wall extending from the base portion, the dividing wall being positioned between the first and second expandable envelopes such that the first and second expandable envelopes are located on opposite sides of the dividing wall, the method preferably further comprises positioning the dividing wall so that it lies below the surface feature or a desired position of the surface feature.

Optionally, the method further comprises allowing the overlying tissue to adhere to the external surface of the tissue expander, which preferably has first and second expandable envelopes having a textured external surface for promoting adhesion to overlying tissue, before commencing expansion.

Preferably, the first and second envelopes are expanded to the same degree for as long as the surface feature is in the desired position, so as to maintain the surface feature in the desired position.

Preferably, the first and second envelopes are inflated to differing degrees for as long as the surface feature is not in the desired position, whereby over-expansion of one envelope is arranged to cause migration of the surface feature towards the under-inflated envelope, by increasing expansion of the tissue overlying the over-expanded envelope.

In certain embodiments, the overlying tissue is a pectoral tissue and the surface feature is a nipple-areola complex.

In a further aspect the invention provides a method of expanding a breast envelope to control the displacement of a nipple-areola complex on the breast envelope as it is being expanded. The method comprises:

implanting sub-pectorally a tissue expander having first and second expandable envelopes located alongside one another, whereby the tissue expander is positioned such that the first expandable envelope is positioned laterally relative to the breast meridian and the second expandable envelope is positioned medially relative to the breast meridian;

monitoring the position of the nipple-areola complex; and

injecting a first volume of biocompatible fluid into the first expandable envelope and injecting a second volume of biocompatible fluid into the second expandable envelope, said first and second volumes being determined on the basis of said monitored position;

whereby the first and second volumes are arranged, via the first and second expandable envelopes respectively, to maintain or migrate the nipple-areola complex in or to a desired position as the breast envelope is expanded.

In yet another aspect the invention provides a kit for expanding a breast envelope to control the displacement of a nipple-areola complex on the breast envelope as it is being expanded. The kit includes a tissue expander implant according to embodiments of the present invention, a form for recording the progress of the expansion procedure and a magnetic device for locating a resealable port. Preferably, the kit also includes a device for measuring the distance of the nipple-areola complex from the mid-line of the patient.

By controlling sideways movement as the tissue expands outwardly the surface feature can be maintained in, or moved to, a desired position. Clearly the expansion process will tend to move the surface feature substantially normal to a plane tangential to the surface feature. Whilst tissue expanders of the present invention will, by definition, cause this type of movement, it is sideways movement approximately in the tangential plane which the tissue expander of the invention seeks to control so that, in plan view, the surface feature ends up at the desired location.

The use of tissue expanders in the group of patients undergoing nipple sparing mastectomies requires the ability to variably expand the sub-muscular pocket if the displaced NAC position is identified pre-operatively or during the expansion process. Such versatility allows for symmetrical NAC position and improved breast aesthetics as the breast-NAC relationship will tend to be maintained and will tend to be symmetrical with the contralateral unoperated breast. For example, if lateral migration of the NAC is detected during the out-patient expansion the expandable envelope on the lateral side of the expander may be overexpanded relative to the expandable envelope on the medial side.

Laterally displaced NAC position may be detected pre-mastectomy due to previous surgery in the lateral aspect of the breast and in this case the differential tissue expander of the invention is arranged to allow for over expansion of the lateral side of the sub-muscular pocket early in the out-patient expansion phase to correct lateral pocket tightness allowing the NAC position to be corrected to the desired NAC position.

The differential tissue expander has applications in other areas of the body where differential tissue expansion is required, such as nasal and ear reconstruction surgery.

### BRIEF DESCRIPTION OF THE DRAWINGS

FIG. 1 shows a partially schematic top plan view of a first embodiment of a differential tissue expander implant of the invention;

FIG. 2 shows a cross-section of the tissue expander implant of FIG. 1;

FIG. 2A shows an enlarged cross-sectional view of the outer domed wall of the tissue expander of FIG. 1;

FIG. 3 shows a top plan view of a second embodiment of a differential tissue expander implant of the invention;

FIG. 4 shows a cross-section of the tissue expander implant of FIG. 3;

FIG. 5 shows a cross-section of the tissue expander implant of FIG. 1 with the lateral envelope over-inflated;

FIG. 6 shows a partially schematic top plan view of a third embodiment of a differential tissue expander implant of the invention;

FIG. 7 shows a partially schematic side view of a fourth embodiment of a differential tissue expander implant of the invention with the lateral envelope over-inflated;

FIG. 8 shows a partially schematic view of the first embodiment of a differential tissue expander implant in position beneath the right pectoralis major for implementing right breast reconstruction; and

FIG. 9 shows a cross-section of the tissue expander of FIG. 8 with the lateral expandable envelope over-inflated.

### DETAILED DESCRIPTION OF THE EMBODIMENTS

In addition to the complications that can occur using tissue expanders in breast reconstruction, the inventor has found that with current tissue expanders in a significant proportion of women who have undergone nipple sparing mastectomies the NAC laterally migrates during the expansion process resulting in a permanent laterally displaced and unaesthetic location.

Asymmetrical sub-muscular pocket expansion, resulting in lateral NAC displacement, is likely due to tightness in the lateral half of the sub-muscular pocket. This tightness in the lateral half of the sub-muscular pocket causes under-expansion of the lateral half of the pocket and relative over-expansion of the more flexible medial half of the sub-muscular pocket, resulting in under-expansion of the skin overlying the lateral half of the sub-muscular pocket, over-expansion of the skin underlying the medial half of the sub-muscular pocket and consequent lateral NAC migration. There are a number of possible causes of tightness in the lateral half of the sub-muscular pocket and the inventor has deduced that these include:
1. previous lateral lumpectomy with resultant loss of skin in the lateral aspect of the breast;
2. scar contracture of the scar resulting from the mastectomy incision lying over the lateral aspect of the sub-muscular pocket;
3. tightness in the suture line between the pectoralis major and serratus anterior muscles;
4. previous radiotherapy to the lateral aspect of the breast following previous lateral lumpectomy;
5. when used in conjunction with a latissimus dorsi reconstruction, tightness as a result of the sutures to the lateral edge of the latissimus dorsi, which are placed to prevent lateral migration of the tissue expander; and
6. incomplete denervation of the latissimus dorsi muscle results in partial latissimus dorsi function which causes a lateral pull on the tissue expander and may cause tightness in the lateral aspect of the sub-muscular pocket.

In addition, medial placement of the tissue expander or displacement of the tissue expander, possibly due to tightness in the lateral half of the pocket, also causes over-expansion of the medial half of the pocket and under-expansion of the lateral half of the pocket resulting in lateral NAC migration.

Further, it has been the inventor's clinical observation over a number of years that the lateral mastectomy scars do not expand as would be expected when using the currently available tissue expanders. This implies that the force exerted by the current tissue expanders is insufficient to expand scar tissue and therefore has a greater effect on non-scar tissue, namely the medial sub-muscular pocket. This confirms the inventor's impression that over-expansion of the medial half of the sub-muscular pocket does occur with the current tissue expanders, resulting in lateral NAC migration.

Lateral NAC migration leads to an unaesthetic appearance in nipple sparing mastectomies which defeats the purpose of initially preserving the NAC if it is ultimately going to lie in an abnormal lateral position. Tightness in the lateral aspect of the pocket has also been noted in skin sparing mastectomies when the NAC has been excised and release of the lateral expander pocket capsule is required at the exchange of the permanent prosthesis to allow a more natural appearance.

When the NAC is excised and a skin sparing mastectomy is undertaken and the patient elects to have a latissimus dorsi and tissue expander/implant reconstruction, a corresponding circle of skin is fashioned during the latissimus dorsi harvest and is used to reconstruct the areola defect when the latissimus dorsi is tunneled to the mastectomy defect. The resultant disc of skin which is used to reconstruct the areola defect is called the areola template. Even in this group of patients the areola template has been noted by the inventor to wander laterally during the tissue expansion process. As a result of this the areola template is in an asymmetrical position compared to the contralateral areola position and therefore cannot be used for subsequent NAC reconstruction.

Digital computer analysis of 25 consecutive expander-based breast reconstructions following nipple sparing mastectomies has been undertaken by the inventor. Thirteen breast reconstructions utilised a sub-muscular tissue expander and the other 12 utilised sub-latissimus dorsi tissue expanders. The same style of expander using an intrinsic port was used and the expander protocol as described was utilised. There were no complications with any of the cases and pre and post-expansion measurements and digital computer analysis of the NAC position was carried out. The results showed that in the sub-muscular tissue expander group the average lateral NAC migration was 19 mm and lateral NAC migration was observed in all cases. In the latissimus dorsi tissue expander group the average lateral NAC migration was 10 mm and no migration was observed in three cases. The extent of the lateral NAC migration appeared to be volume dependant so that women with larger breasts who require a greater volume tended to have more lateral NAC migration. However, less lateral NAC migration was observed during the expansion phase when a lateral NAC position was observed pre-mastectomy.

Overall 88% of cases showed lateral migration of the NAC with 100% of the sub-muscular group and 75% of latissimus dorsi group showing lateral migration. Aesthetically, migration of even less than 10 mm in a unilateral reconstruction results in a significant and deleterious difference in appearance.

It has accordingly been recognised by the inventor that the current range of commercially available tissue expanders cannot compensate for tightness of the lateral half of the pocket and resultant over-expansion of the medial half of the sub-muscular pocket which leads to lateral NAC migration. Such tissue expanders do not have the flexibility to counteract lateral NAC migration when it is detected during the expansion process, nor can they correct NAC asymmetry present prior to a mastectomy.

Currently there is no expander available that has the flexibility to allow correction of lateral NAC migration once it has occurred. In a number of cases due to the previous surgery in the lateral aspect of the breast prior to mastectomy, the NAC is pre-operatively lying in a lateral position compared to the un-operated side and current expanders only worsen the lateral deviation.

In addition to breast reconstruction surgery, tissue expanders are also used in other areas of the body where reconstruction is required. These tissue expansion processes can also be affected by migration of surface features, for example, in ear reconstruction surgery, which can often require the insertion of a tissue expander beneath the ear. In these cases, it is aesthetically important that the ear subject to reconstruction maintains a symmetrical position with regard to the unoperated ear and that no migration occurs. Similarly, in nasal reconstruction, it is aesthetically important to ensure no migration of the nose occurs. Such surface feature migration can be caused by, amongst other reasons, skin loss on one side of the surface feature as a result of previous surgery and scar contracture from an incision on one side of the surface feature.

As previously mentioned, current tissue expanders do not have the flexibility to counteract migration of surface features when migration is detected.

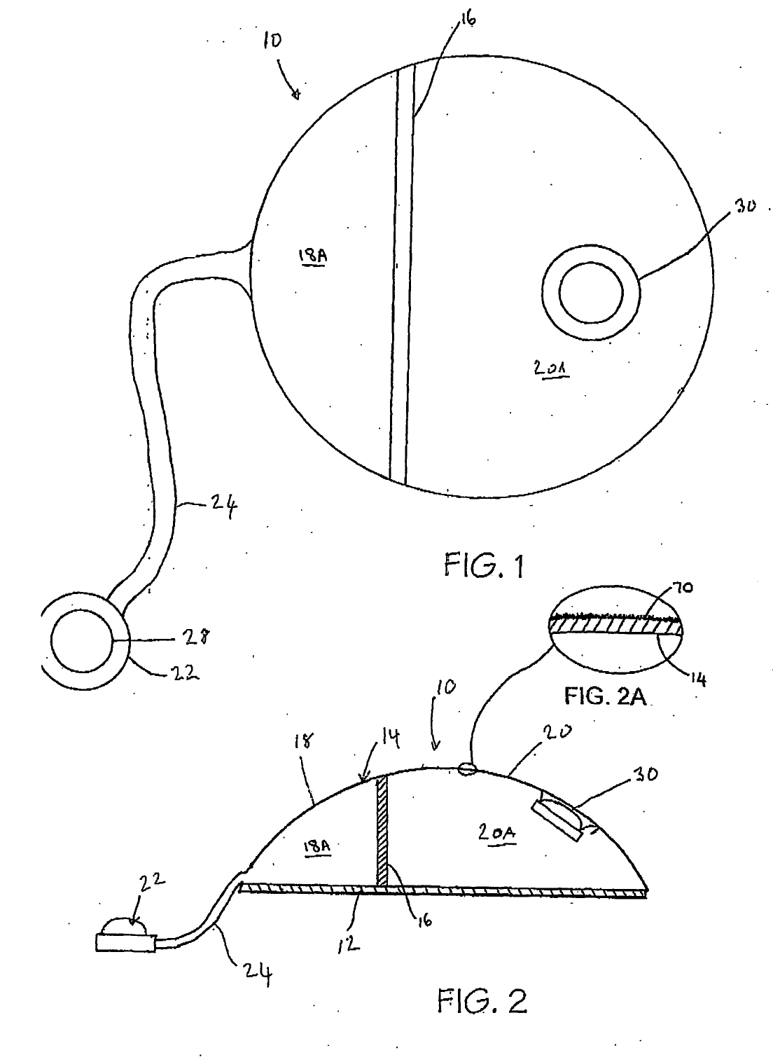

Referring now to FIGS. 1 and 2, a first embodiment of a tissue expander breast implant 10 comprises a base silicone wall 12 which is substantially circular in form. An outer domed wall 14 extends upwardly from the outer periphery of the base silicone wall 12, and is formed from an elastomeric silicone material. A laterally offset dividing wall 16 formed from a non-expandable silicone sheet extends upwardly at right angles from the base silicone wall 12 to the outer wall 14 so as to provide first or lateral and second or medial adjacent envelopes 18 and 20 defining respective first and second chambers 18A and 20A. A remote resealable injection valve or port 22 communicates with the chamber of the lateral envelope 18 via siloxane polymer tubing 24, and the medial envelope 20 is formed with an integral resealable injection valve or port 30. The remote valve 22 is in the form of a siloxane polymer dome having a posterior metal disc 28 to prevent needle perforation through the posterior aspect of the remote valve 22.

The non-expandable dividing wall 16 ensures that the two expandable envelopes 18 and 20 are expandable independently of one another. Expandable independently of one another, as used in this specification, means that the inflation of either envelope has no material effect on the movement, shape or configuration of the other envelope. This includes, having no material effect on the volume of the other envelope or the projection of the other envelope from the base wall and the dividing wall. It will be appreciated that the dividing wall needs to be made less expandable than a combination of the expandable envelope and the overlying tissue, with the result that an increase in volume of the chamber will translate into outward deformation of the envelope and the overlying tissue rather than the dividing wall and the base wall.

The outer surface of the tissue expander 10 is textured to facilitate adhesion of the underlying and overlying muscle to the tissue expander 10. The textured surface 70 of the outer domed wall 14 is depicted in FIG. 2A. Adhesion of the muscle to the tissue expander 10 provides greater control over the expansion of the overlying muscle and overlying skin as a result of:
1 the tissue expander 10 being held more securely in position and conforming shape within the sub-muscular pocket;
2 the tissue expander 10 more effectively applying a force to the adhered overlying muscle; and
3 each of the expandable envelopes 18 and 20 selectively expanding that portion of the overlying muscle which directly adheres to that expandable envelope.

Texture can be applied to the surface of the tissue expander 10 by methods known in the art, which include providing a sandy or gritty lining in the expander mould.

The dividing wall 16 of the tissue expander 10 is laterally offset in order to reflect the laterally offset position of the NAC and the breast meridian. Generally, the dividing wall 16 will be positioned at approximately one third of the length of the diameter or width of the tissue expander from the lateral edge of the base silicone wall 12. The dividing wall 16 is preferably placed below the NAC to enable selective expansion of the tissue on either side of the NAC, depending on the direction of migration.

The tissue expander 10 is made in a variety of different sizes and base diameters so as to achieve the correct biodimensional outcome for women having differently sized breasts. The mathematical relationship between the base diameter, the height and the projection of the tissue expander will generally be the same as for prior art tissue expanders.

The base wall 12, expandable envelopes 18 and 16 and dividing wall 16 of the tissue expander 10 are made from the same elastomeric silicone material, but with different thicknesses. The expandable envelopes 18 and 20 have thinner walls than the base wall 12 and dividing wall 16, such that the envelopes 18 and 20 are expandable and the base wall 12 and dividing wall 16 are not expandable in ordinary use. For example, the operative walls of the expandable envelopes 18 and 16 are 0.5-1 mm thick and the dividing wall 16 and base wall 14 are 2-3 mm thick.

The silicone polymer tubing 24 extends laterally from the lateral envelope 18 to help prevent rotation of the axis of the tissue expander 10. Sutures are placed on either side of the tubing 24 so that it anchors the base wall 12 and helps prevents rotation of the axis of the tissue expander 10, in particular before the surrounding tissue has adhered to the textured surface of the tissue expander 10.

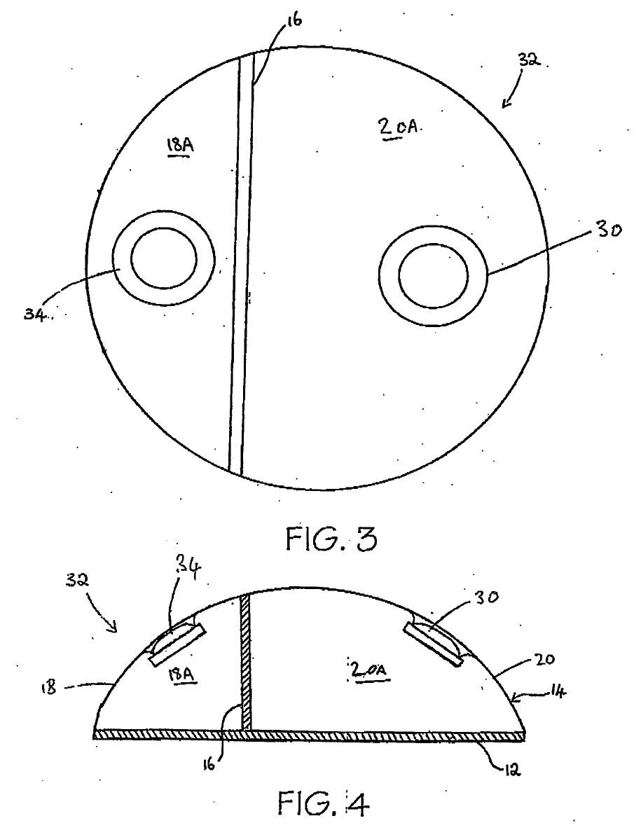

Referring now to FIGS. 3 and 4, a second embodiment of a tissue expander breast implant 32 is shown which is essentially identical to the first embodiment, save that the lateral envelope 18 is also formed with an intrinsic valve 34.

In these particular embodiments, the lateral envelope 18 is arranged to project outwardly further than the medial envelope 20 by having a greater pressure in the lateral envelope 18 than the medial envelope 20. FIG. 5 shows the first embodiment of the tissue expander 10 with the lateral envelope over-inflated. In this particular embodiment, the over-inflation of the lateral envelope 18 allows for the correction of the drift of an overlying NAC in the more commonly encountered lateral direction.

Alternatively, the greater projection of the lateral envelope 18 can be as a result of the outer wall 14 of the lateral envelope 18 being formed from substantially more material than would normally be required to form the outer wall 14 of the lateral envelope 18. As a result, when the tissue expander 10 is deflated, the greater surface area of the outer wall 14 of the lateral envelope 18 would result in the lateral envelope 18 having a folded, baggy or wrinkled appearance.

Alternatively or in conjunction with the above arrangements, the greater projection of the lateral envelope 18 can possibly be achieved by the outer wall 14 of the lateral envelope 18 having a greater co-efficient of elasticity than the outer wall 14 of the medial envelope 20.

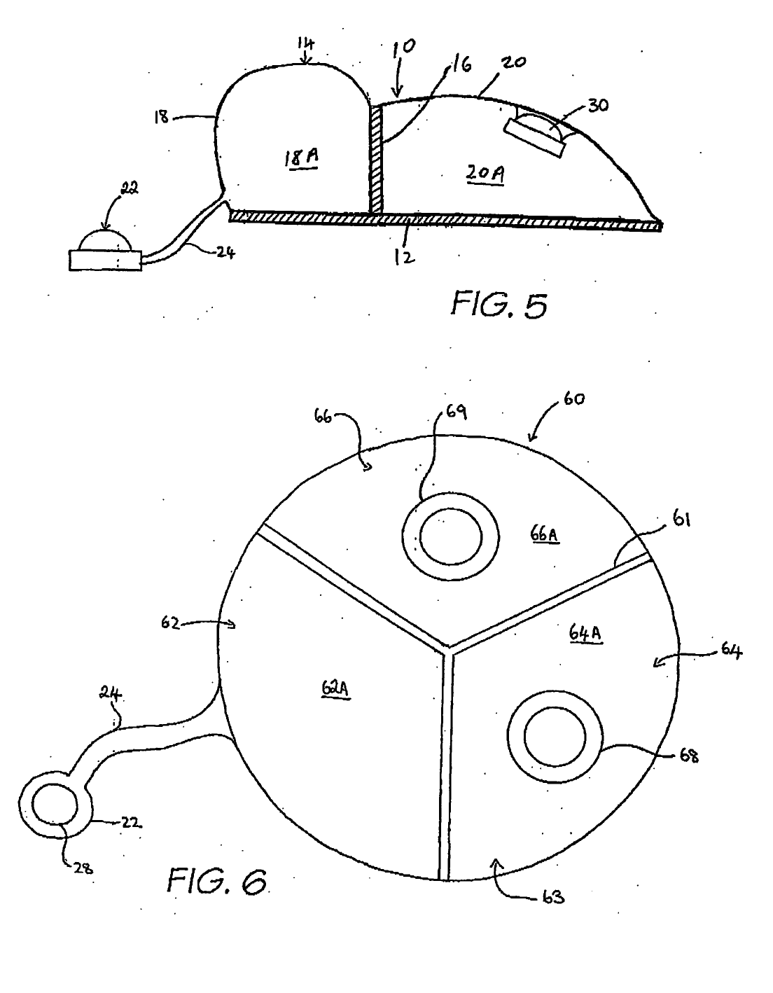

Referring now to FIG. 6, a third embodiment 60 of a tissue expander breast implant comprises a base silicone wall (not shown) which is substantially circular in form. A domed outer wall 63 extends upwardly from the outer periphery of the base silicone wall, and is formed from a textured elastomeric silicone material. A dividing wall 61 formed from a non-expandable silicone sheet extends upwardly at right angles from the base silicone wall to the outer wall 63. The dividing wall 61 is formed in a Y-shape. The dividing wall 61, the base silicone wall and the outer wall 63 form first or lateral, second or medial and third or superior adjacent envelopes 62, 64 and 66 respectively, defining respective first, second and third chambers 62A, 64A and 66A. A remote resealable injection valve or port 22 communicates with the chamber 62A of the lateral envelope 62 via siloxane polymer tubing 24, and the medial and superior envelopes 64 and 66 are each formed with one integral resealable injection valve or port 68 and 69 respectively. The remote valve 22 is the same as described for FIG. 1.

The tissue expander 60 allows the controlled differential expansion of tissue surrounding a surface feature situated above the junction of the dividing wall 61 in multiple directions by selective expansion of one or more of the chambers 62A, 64A and 66A.

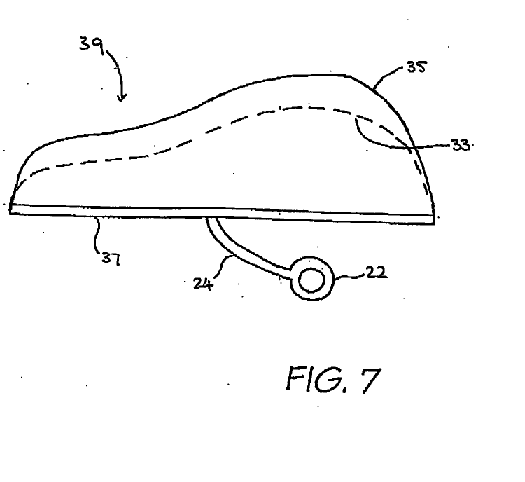

It will also be appreciated that a differential tissue expander breast implant can be made in shapes which more realistically replicate the appearance of a natural breast. For example, a fourth embodiment, depicted in FIG. 7, is a tissue expander 39 formed with a teardrop-shaped base silicone wall 37. The dividing wall 33 and lateral envelope 35 also reflect the teardrop-shape, with the height of the dividing wall 33 and lateral envelope 35 increasing at the wider end of the base silicone wall 37. The tissue expander 39 is to be positioned in a patient so that the larger end of the tissue expander 39 forms the lower portion of a breast. These more biocompatible shapes will generally be the same as for the prior art.

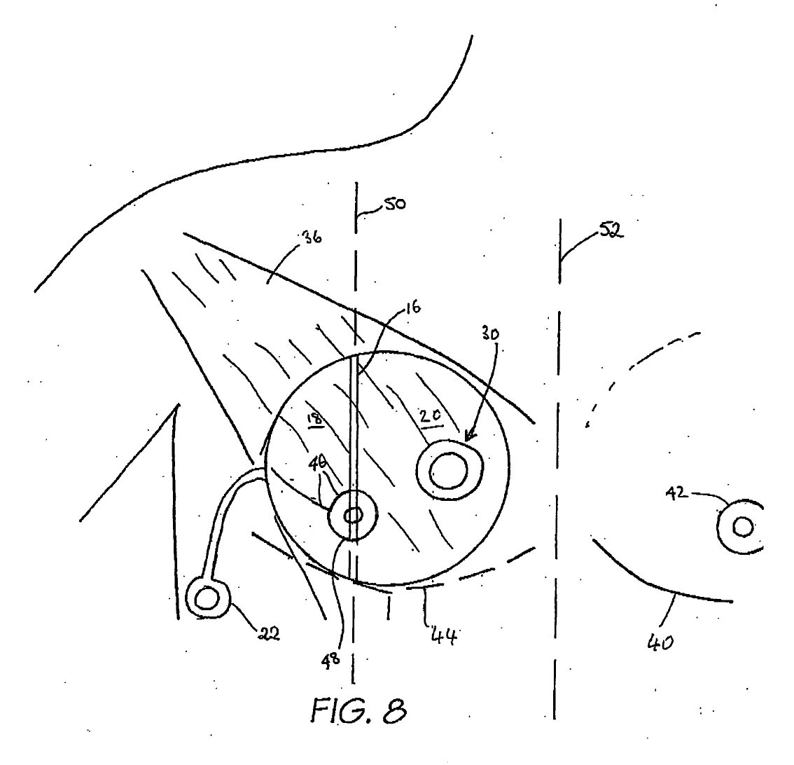

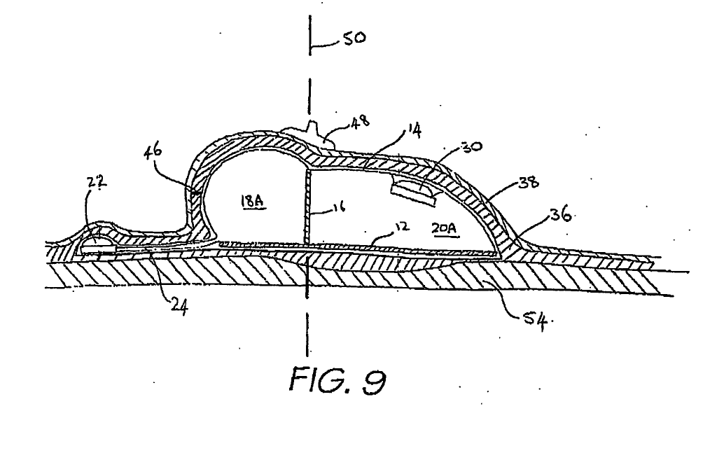

In FIGS. 8 and 9, the tissue expander 10 of FIG. 1 is shown in position in a pocket beneath the right pectoralis major muscle 36 for reconstruction of the right breast 38. The left breast 40 and left NAC 42 are shown for comparison purposes, as is the previous infra-mammary fold 44 of the right breast 38. Also indicated is the nipple sparing mastectomy incision 46 and the preserved right NAC 48.

The tissue expander 10 is inserted into the sub-pectoral pocket in such a way that the dividing wall 16 of the tissue expander 10 is placed in line with the breast meridian 50 which runs vertically through the right breast 38 beneath the NAC 48. The tissue expander 10 is placed sub-pectoralis major at the time of the nipple sparing mastectomy. The inner surface muscle fibres of the muscle forming the sub-muscular pocket spontaneously adhere to the textured surface of the tissue expander 10 as they heal from the trauma associated with the dissection of the pectoralis major.

The expansion protocol involves pre-operative measurements of the NAC 48 position. At each expansion visit, the NAC 48 position is measured from the mid-line 52 with a tape measure which has a marking of the pre-operative position noted on it, as well as the desired NAC position, if this does not coincide with the pre-operative position. The tape measure also extends beyond the mid-line 52 to the contra-lateral NAC 42 such that the NAC 48 position on the reconstructed site can be monitored and aimed to be kept in the same symmetrical position as the unoperated side owing to the differential tissue expansion effect.

Volumes of saline proportional to the total volume of each envelope 18 and 20 are initially injected through each port 22 and 30. When lateral NAC migration is detected over-inflation of the lateral envelope 18 of the tissue expander 10 as shown in FIG. 9 will be undertaken to correct the lateral migration and keep the NAC 48 in a symmetrical position with the contra-lateral side. This will prevent over expansion of the medial skin as the tissue expander 10 will allow each hemisphere of the sub-muscular pocket to be selectively expanded in a graded fashion to allow the NAC 48 to remain in the same position as was noted pre-operatively.

Alternatively, the tissue expander 10 can be used to correct NAC 48 asymmetry in relation to the unoperated side present pre-operatively, typically due to previous surgery performed on the breast 38, such as a lumpectomy.

For example, if the NAC 48 is laterally displaced before tissue expansion begins, the lateral envelope 18 can be over-inflated from the beginning of the tissue expansion process in order to correct the lateral displacement of the NAC 48. This will prevent over expansion of the medial skin as the tissue expander 10 will again allow each hemisphere of the sub-muscular pocket to be selectively expanded in a graded fashion to correct the lateral displacement of the NAC 48.

Once the appropriate volume has been attained and the NAC 48 position is symmetrical the differential tissue expander 10 is removed and a customised permanent prosthesis which matches the dimensions of the asymmetrical sub-muscular pocket which was formed by the tissue expander 10 is placed within the sub-muscular pocket, thereby achieving an improved aesthetic result.

Referring to FIG. 9, this depiction of the tissue expander 10 in situ incorporates a rib 54 and the overlying epidermal tissue 38.

It will be appreciated that in many cases (and in the experience of the inventor this could be as high as 50-75%) the NAC 48 is, post-operatively, not significantly displaced. As a result, the expansion regime could involve simply maintaining the NAC in the desired position by injecting proportionally the same volumes of fluid into the lateral and medial expandable envelopes 18 and 20. When following the typical expansion protocol previously described, of injecting 60 ml of saline twice a week, this would involve injecting 20 ml of fluid into the lateral envelope 18 and 40 ml of fluid into the medial envelope at 20 at the bi-weekly intervals. This would achieve uniform expansion of the tissue expander with the result that both the lateral and medial portions of the overlying tissue will be evenly expanded. This uniform expansion is in contrast to the expansion of a prior art single chamber expander which undergoes deformation as a result of the generally increased resistance of the overlying tissue of the lateral side of the sub-muscular pocket. This deformation results in greater expansion of the medial side of the expander, hence and resultant lateral displacement of the NAC.

The inventor performed a comparative trial using a dual chambered tissue expander of an embodiment of the present invention having a textured surface and two chambers of equal volume (dual chamber expander) and a prior art single chambered tissue expander also having a textured surface (single chamber expander).

The test subject of the comparative trial was a greyhound. The single chamber expander and the dual chamber expander were positioned over the thoracic cage on the right and left sides of the test subject respectively.

The tissue expanders were implanted sub-muscularly and a nipple-areola template (NAT) was implanted above the centre of each tissue expander. The dual chamber expander was orientated so that the inelastic central wall extended towards the NAT from the base of the tissue expander directly beneath the NAT. A caudal incision was made extending from the caudal edge of the NAT for approximately the same distance as an incision required when performing a nipple sparing mastectomy. As a result, the scar caused by the incision was situated above the caudal side of each expander. Such an arrangement allows the caudal and cranial sides of the tissue expanders to be equated to the lateral and medial sides respectively of a tissue expander implanted in a woman who has previously undergone a nipple sparing mastectomy.

The position of each NAT was determined by reference to a cranial fixed bony landmark on the relevant side of the test subject. The cranial fixed bony landmark was the posterior edge of the scapula while the test subject was standing with its front legs vertical.

When implanted into the test subject, the single chamber expander contained 60 ml of saline and the dual chamber expander contained 60 ml of saline, 30 ml in each chamber. Three weeks after the expanders had been implanted, the expansion protocol was commenced. Generally, each week the position of each NAT relative to its cranial fixed bony landmark was measured and then the tissue expanders were injected with saline. Turning point measurements of migration are also recorded.

The 6 week expansion protocol is tabulated in Table 1. The date of implant of the expanders is represented as time 0 and the date of injection represented in weeks after the date of implant of the expanders.

TABLE 1

| | | | Dual chamber expander | | |
|---|---|---|---|---|---|
| | Single chamber expander | | Volume of | | |
| | Volume of | | saline added | | |
| Time | saline | Migration | Caudal | Cranial | Migration |
| (week) | added | of NAT | chamber | chamber | of NAT |
| 0 | 60 ml | N/A | 30 ml | 30 ml | N/A |
| +3 | 60 ml | N/A | 30 ml | 30 ml | N/A |
| +4 | 60 ml | | 30 ml | 30 ml | |
| +5 | 60 ml | | 30 ml | 30 ml | |
| +6 | 60 ml | 5 mm | 40 ml | 20 ml | 5 mm |
| +7 | 60 ml | | 40 ml | 20 ml | |
| +8 | 60 ml | | 40 ml | 20 ml | |
| +9 | — | 10 mm | — | — | 0 mm |
| Total | 420 ml | | 240 ml | 180 ml | |
| saline | | | | | |

N/A—not applicable

Before the injection in week +3 the NAT overlying the single chamber expander was 100 from its cranial fixed bony landmark and the NAT overlying the dual chamber expander was 90 mm from its cranial fixed bony landmark.

The measurements taken at week +6 confirmed an early caudal migration of the NATs overlying both the single and dual chamber expanders. In response to the slight 5 mm migration of the NAT overlying the dual chamber expander the expansion regime was altered so that the caudal chamber was injected with twice the volume of saline as week.

At week +9, the end of the expansion protocol, each tissue expander held 420 ml of saline. In the dual chamber expander 240 ml was contained in the caudal chamber and 180 ml was contained in the cranial chamber.

At week +9 the NAT overlying the single chamber expander was situated 110 mm from the cranial fixed bony landmark, evidencing a migration of 1 cm during the expansion process. In contrast, at week +9 the early migration of the NAT overlying the dual chamber expander had been corrected and so the NAT was again situated 90 mm from the cranial fixed bony landmark,

It was concluded, on the basis of the above results, that the overexpansion of the caudal chamber of the dual chamber expander maintained the NAT overlying the dual chamber expander in its original position 90 mm from the cranial fixed bony landmark.

Embodiments of the tissue expander of the present invention may be supplied in a kit including a tabulated form, including tables similar to that indicated in Tables 2 and 3. A pro form a version of the form is set out below for enabling plastic and reconstructive surgeons to monitor the progress of their patients. Allowance is made for nipple sparing mastectomies on both breasts. It is clear from the form that allowance is made for pre-operative breast volume and NAC position as well as desired breast volume and NAC position. Allowance is also made for entry of volumetric and NAC migration details at bi-weekly intervals for a period, say, of four weeks.

It will be understood that the invention disclosed and defined herein extends to all alternative combinations of two or more of the individual features mentioned or evident from the text or drawings. All of these different combinations constitute various alternative aspects of the invention.

The foregoing describes embodiments of the present invention and modifications, obvious to those skilled in the art can be made thereto, without departing from the scope of the present invention.

Reference to any prior art in the specification is not, and should not be taken as, an acknowledgement or any form of suggestion that this prior art forms part of the common general knowledge in Australia or any other jurisdiction.

TABLE 2

| | Left | Right |
|---|---|---|
| | breast | breast |
| Actual pre-operative volume (ml) | | |
| Actual pre-operative distance of NAC from the | | |
| midline (cm) | | |
| Actual post-operative volume (ml) | | |
| Actual post-operative distance of NAC from the | | |
| midline (cm) | | |
| Desired volume (ml) | | |
| Desired distance of NAC from the midline (cm) | | |

TABLE 3

| | | Left breast | | | | Right breast | | | |
|---|---|---|---|---|---|---|---|---|---|
| | | Volume of saline | | | | Volume of saline | | | |
| | | added (ml) | | Accumulated | Distance of NAC | added (ml) | | Accumulated | Distance of NAC |
| Week | Date | Lateral | Medial | volume (ml) | from mid-line (cm) | Lateral | Medial | volume (ml) | from mid-line (cm) |
| 0 | | | | | | | | | |
| 0.5 | | | | | | | | | |
| 1.0 | | | | | | | | | |
| 1.5 | | | | | | | | | |
| 2.0 | | | | | | | | | |
| 2.5 | | | | | | | | | |
| 3.0 | | | | | | | | | |
| 3.5 | | | | | | | | | |
| 4.0 | | | | | | | | | |
| Final | | | | | | | | | |

1. A tissue expander implant arranged to be anchored in position within a cavity formed in the body for the controlled expansion of overlying tissue, the tissue expander implant comprising a first expandable envelope defining a first closed chamber, a first inflation interface for enabling the controlled inflation of the first expandable envelope with a biocompatible fluid, a second expandable envelope located in side-by-side relationship with the first expandable envelope and defining a second closed chamber and a second inflation interface for enabling the controlled inflation of the second expandable envelope with a biocompatible fluid, the first and second expandable envelopes being differentially expandable independently of one another to enable the tissue overlying the first and second expandable envelopes to be expanded to differing degrees, thereby to maintain, or move, at least one surface feature of the overlying tissue in, or to, a desired position.

2. A tissue expander implant according to claim 1 further comprising a base portion and a dividing wall extending from the base portion, the dividing wall being positioned between the first and second expandable envelopes such that the first and second expandable envelopes are located on opposite sides of the dividing wall.

3. A tissue expander implant according to claim 2 wherein the dividing wall is inelastic relative to the operatively outermost walls of the first and second expandable envelopes.

4. A tissue expander implant according to claim 2, wherein the dividing wall is common to, and partially defines, both the first and second adjacent closed chambers.

5. A tissue expander implant according to claim 2 wherein the base portion partially defines the first and second adjacent closed chambers.

6. A tissue expander implant according to claim 2 wherein the dividing wall is arranged to be positioned beneath the at least one surface feature, or a desired location of the at least one surface feature, with the first and second expandable envelopes being arranged to be located beneath and substantially on opposite sides of the at least one surface feature and being configured to expand the overlying tissue to a desired degree when in said position.

7. A tissue expander implant according to claim 1 wherein one of the first or second expandable envelopes is configured to have a larger volume than the other.

8. A tissue expander implant according to any claim 1 wherein the external tissue-contacting surface of the expander is textured for promoting adhesion to the tissue surrounding the tissue expander implant.

9. A tissue expander implant according to claim 2 wherein the dividing wall is off-centre in relation to the base portion to define the first and second expandable envelopes having differing volumes.

10. A tissue expander implant according to claim 9 wherein the dividing wall is positioned approximately one third of the breadth of the base portion from one edge of the base portion.

11. A tissue expander implant according to claim 1, which is a breast expander implant, wherein the overlying tissue is mammary tissue and the surface feature is a nipple-areola complex, whereby the first and second expandable envelopes are configured to undergo controlled expansion independently of one another via the first and second inflation interfaces respectively to control sideways displacement of the nipple-areola complex.

12. A tissue expander implant according to claim 1 wherein the first and second inflation interfaces are resealable injection valves or ports.

13. A tissue expander implant according to claim 12 wherein the resealable injection valves or ports are integral with the first and second expandable envelopes.

14. A tissue expander implant according to claim 12 wherein one of the resealable injection valves or ports is remote from either the first or second expandable envelope and the other resealable injection valve or port is integral with the other expandable envelope.

15. A tissue expander implant according to claim 2 wherein the expander implant is integrally moulded from an elastomeric material and the relative elasticity of the first and second expandable envelopes, the dividing wall and the base portion are determined by their relative thicknesses, with the dividing wall and the base portion being thicker than the first and second expandable envelopes.

16. A tissue expander implant according to claim 15 wherein the elastomeric material is a silicone elastomer.

17. A tissue expander implant according to claim 1 further comprising a third expandable envelope defining a third closed chamber, a third inflation interface for enabling the controlled inflation of the third expandable envelope with a biocompatible fluid, wherein the third expandable envelope is located in a side-by-side relationship with at least one of the first and second expandable envelopes and is expandable independently of the first and second expandable envelopes.

18. A tissue expander implant according to claim 17 further comprising a fourth expandable envelope defining a fourth closed chamber, a fourth inflation interface for enabling the controlled inflation of the fourth expandable envelope with a biocompatible fluid, wherein the fourth expandable envelope is located in a side-by-side relationship with at least one of the first, second or third expandable envelopes and is expandable independently of the first, second and third expandable envelopes.

19. A method of expanding tissue to control the displacement of a surface feature on the tissue as it is being expanded, the method comprising:
implanting beneath a section of tissue which is arranged to be expanded a tissue expander implant having first and second expandable envelopes located alongside one another;
monitoring the position of the surface feature on the tissue; and
independently expanding the first and second expandable envelopes in response to said monitoring to maintain or migrate the surface feature in or towards a desired position as the overlying tissue is expanded.

20. A method of expanding tissue according to claim 19 further comprising repeatedly monitoring the position of the surface feature and repeatedly independently expanding the first and second expandable envelopes so that the desired position of the surface feature at a desired degree of expansion of the tissue is attained.

21. A method of expanding tissue according to claim 20 further comprising establishing a desired final position of the surface feature and a desired degree of final expansion.

22. A method of expanding tissue according to claim 19 wherein the tissue expander further comprises a base portion and a dividing wall extending from the base portion, the dividing wall being positioned between the first and second expandable envelopes such that the first and second expandable envelopes are located on opposite sides of the dividing wall, and the method further comprises positioning the dividing wall so that it lies below the surface feature or a desired position of the surface feature.

23. A method of expanding tissue according to claim 19 wherein the first and second expandable envelopes have a textured external surface for promoting adhesion to overlying tissue, and the method further comprises allowing the overlying tissue to adhere to the external surface before commencing expansion.

24. A method of expanding tissue according to claim 19 wherein the first and second envelopes are expanded to the same degree for as long as the surface feature is in the desired position, so as to maintain the surface feature in the desired position.

25. A method of expanding tissue according to claim 19, wherein the first and second envelopes are differentially inflated to cause migration of the surface feature away from a position overlying an originating envelope towards a position overlying a destination envelope, whereby inflation of the originating envelope relative to the destination envelope causes migration of the surface feature towards the position overlying the destination envelope, by increasing expansion of the tissue overlying the originating envelope relative to the tissue overlying the destination envelope.

26. A method of expanding tissue according to claim 19 wherein the overlying tissue is a pectoral tissue and the surface feature is a nipple-areola complex.

27. A method of expanding a breast envelope to control the displacement of a nipple-areola complex on the breast envelope as it is being expanded, the method comprising:
implanting sub-pectorally a tissue expander having first and second expandable envelopes located alongside one another, whereby the tissue expander is positioned such that the first expandable envelope is positioned laterally relative to the breast meridian and the second expandable envelope is positioned medially relative to the breast meridian;
monitoring the position of the nipple-areola complex; and
injecting a first volume of biocompatible fluid into the first expandable envelope, and injecting a second volume of biocompatible fluid into the second expandable envelope, said first and second volumes being determined on the basis of said monitored position;
whereby the first and second volumes are arranged, via the first and second expandable envelopes respectively, to maintain or migrate the nipple-areola complex in or to a desired position as the breast envelope is expanded.

28. A method of expanding a breast envelope according to claim 23 further comprising repeatedly monitoring, injecting and allowing the mammary tissue to respond until the desired volume of the mammary tissue is obtained.

29. (canceled)

30. (canceled)
